(19) United States
(12) Patent Application Publication
(10) Pub. No.: US 2026/0288232 A1
(43) Pub. Date: Sep. 24, 2026

(54) READING ASSISTANCE DEVICE AND METHOD FOR STIMULATING INTEREST IN READING

(71) Applicant: PCNC Co., Ltd, Gyeonggi-do (KR)

(72) Inventors: Duck Ki KIM, Seoul (KR); So Yeon HEO, Gyeonggi-do (KR)

(21) Appl. No.: 18/874,277

(22) PCT Filed: Oct. 24, 2024
(86) PCT No.: PCT/KR2024/016300

§ 371 Date: Dec. 12, 2024

(30) Foreign Application Priority Data
Nov. 9, 2023 (KR) 10-2023-0154111

(51) Int. Cl. G06F 3/01; G06F 3/16; G06T 11/60; G06V 30/30; G06V 40/18; G09B 17/00

(52) U.S. Cl. CPC G06F 3/013; G06F 3/167; G06T 11/60; G06V 30/30; G06V 40/18; G09B 17/006; G06V 2201/131

(57) ABSTRACT

Proposed are reading assistance device and method for stimulating interest in reading. The reading assistance device includes smart glasses that are worn by a user and capture images of a book which the user is reading and the pupils of the user who is reading to generate corresponding image information, and a controller that detects book-related information and a portion of the book that the user is currently reading on the basis of the generated image information, determines that a preset event has occurred when contents of the detected portion of the book include a preset word, and controls at least one of a visual effect and an auditory effect corresponding to the event to be output.

### TECHNICAL FIELD

The present disclosure relates to a reading assistance device and, more specifically, to reading assistance device and method for stimulating interest in reading which help a user feel interested in reading by generating various effects depending on a reading environment.

### BACKGROUND ART

Recently, children are gradually losing interest in reading as they are exposed to games and animations from a young age. As a result, the problem occurs that children are entering school and entering their growth period without developing the educational benefits of reading, such as literacy, thinking skills, and creativity, due to insufficient reading.

In other words, the problem occurs that children are entering adolescence without having developed the intellectual abilities necessary for early childhood due to a lack of reading training necessary to improve intellectual abilities.

Therefore, there is a need for a way to encourage children to read and naturally increase their reading amount.

### DISCLOSURE

### Technical Problem

An objective that the present disclosure seeks to achieve is to provide reading assistance device and method for stimulating interest in reading which help a user feel interested in reading by providing the user with visual and auditory effects corresponding to events occurring during a reading process.

### Technical Solution

In order to accomplish the above objectives, a reading assistance device according to the present disclosure includes: smart glasses that are worn by a user and capture images of a book which the user is reading and the pupils of the user who is reading to generate corresponding image information; and a controller that detects book-related information and a portion of the book that the user is currently reading on the basis of the generated image information, determines that a preset event has occurred when contents of the detected portion of the book include a preset word, and controls at least one of a visual effect and an auditory effect corresponding to the event to be output.

In addition, the controller may detect status information about a gaze and a focus of the user on the basis of the image information of the pupils of the user whose image is captured, calculate a reading-related concentration level by using the detected status information, and determine that the event has occurred when the calculated concentration level is lower than a preset reference.

In addition, the controller may further detect a reading pattern of the user and whether the user has dyslexia by using the status information, and may support chatbot conversation in conjunction with the smart glasses on the basis of the detected concentration level, the reading pattern, and whether the user has dyslexia.

In addition, when the user reads the book by producing sound, the controller may detect information related to at least one of pronunciation, a breathing position, reading speed, and a misread portion included in voice information about the sound, and may provide feedback information on a part requiring correction on the basis of the detected information.

In addition, when reading an e-book that provides contents of the book in an image form, the controller may replace a name of at least one character of characters included in the contents of the book with a random name and may control the contents of the e-book to be output with the replaced name.

In addition, whenever the random name is output in the contents of the e-book, the controller may determine that the event has occurred.

In addition, the controller may perform analysis of the contents of the book, use a result of the analysis to estimate a personality of each of the characters, and, when one of the characters is selected, control a personality of the corresponding character to be reflected in a chatbot conversation pattern.

In addition, when a user input is input from the user and the user input includes a specific word, the controller may allow the e-book to return to a preset page thereof.

In addition, the controller may determine that a reading completion event has occurred when the detected portion of the book is a last page of the book, and may control a congratulatory message and information about a process of the reading to be output.

In addition, the controller may control the reading completion event to occur only when a record of the user reading the book sequentially from a first page of the book to the last page thereof is saved.

A reading assistance method performed by a reading assistance device according to the present disclosure includes: generating corresponding image information by capturing a book being read by a user and the pupils of the user reading the book; detecting book-related information and a portion of the book that the user is currently reading on the basis of the generated image information; and outputting at least one of a visual effect and an auditory effect corresponding to a preset event by determining that the event has occurred when contents of the detected portion of the book include a preset word.

A reading assistance system according to the present disclosure includes: at least one reading assistance device that assists a reading process so that a user is interested in reading; and a management server that receives user information of the user and monitoring information about the reading process the user from the reading assistance device and manages reading habit of the user by using the received monitoring information, wherein the reading assistance device includes: smart glasses that are worn by the user and capture images of a book which the user is reading and the pupils of the user who is reading to generate corresponding image information; and a controller that detects book-related information and a portion of the book that the user is currently reading on the basis of the generated image information, determines that a preset event has occurred when contents of the detected portion of the book include a preset word, and controls at least one of a visual effect and an auditory effect corresponding to the event to be output.

In addition, the management server may process the monitoring information on the basis of the user information to generate a new story in which the user is included as a character, and may generate image-based content by using the generated story.

In addition, when the user reads the book by producing sound, the controller may detect information related to at least one of pronunciation, a breathing position, reading speed, and a misread portion included in voice information about the sound, and may provide feedback information on a part requiring correction on the basis of the detected information.

In addition, when reading an e-book that provides contents of the book in an image form, the controller may replace a name of at least one character of characters included in the contents of the book with a random name and may control the contents of the e-book to be output with the replaced name.

### Advantageous Effects

According to an embodiment of the present disclosure, an event occurring during a reading process can be determined by using image information obtained by capturing the reading process of a user, and when the event occurs, at least one of a visual effect and an auditory effect corresponding to the event can be output to provide the user with interest in reading.

This can encourage continuous reading activity, thereby improving the literacy, thinking skill, and creativity of a user.

### DESCRIPTION OF DRAWINGS

FIG. 1 is a configuration diagram illustrating a reading assistance system according to an embodiment of the present disclosure.

FIG. 2 is a block diagram illustrating the reading assistance device according to an embodiment of the present disclosure.

FIG. 3 is a block diagram illustrating a management server according to an embodiment of the present disclosure.

FIG. 4 is a flowchart illustrating a reading assistance method according to an embodiment of the present disclosure.

FIG. 5 is a flowchart illustrating a reading assistance method according to another embodiment of the present disclosure.

FIG. 6 is a block diagram illustrating a computing device according to an embodiment of the present disclosure.

### MODE FOR INVENTION

Below, with reference to the attached drawings, embodiments of the present disclosure are described in detail so that those skilled in the art to which the present disclosure belongs can easily implement the present disclosure. However, the present disclosure may be implemented in many different forms and is not limited to the embodiments described herein. In addition, in order to clearly describe the present disclosure in the drawings, parts that are not related to the description have been omitted, and similar parts have been given similar reference numerals throughout the specification.

In this specification and drawings (hereinafter, referred to as “this specification”), duplicate descriptions of identical components are omitted.

In addition, in this specification, when it is said that a component is “connected” or “coupled” to another component, it should be understood that it may be directly connected or coupled to the another component, but there may be other components therebetween. On the other hand, when it is said in this specification that a component is “directly connected” or “directly coupled” to another component, it should be understood that there are no other components therebetween.

In addition, terms used herein are for the purpose of describing particular embodiments only and are not intended to limit the present disclosure.

In addition, in this specification, singular expressions may include plural expressions unless the context clearly indicates otherwise.

In addition, in this specification, it should be understood that terms such as “include” or “have” are intended only to specify the presence of a feature, number, step, operation, component, part, or combination thereof described in the specification, and do not exclude in advance the possibility of the presence or addition of one or more other features, numbers, steps, operations, components, parts, or combinations thereof.

In addition, in this specification, the term “and/or” includes combination of a plurality of listed items or any item of the plurality of listed items. In this specification, “A or B” may include “A”, “B”, or “both A and B”.

In addition, in this specification, detailed descriptions of the disclosed functions and configurations that may obscure the gist of the present disclosure will be omitted.

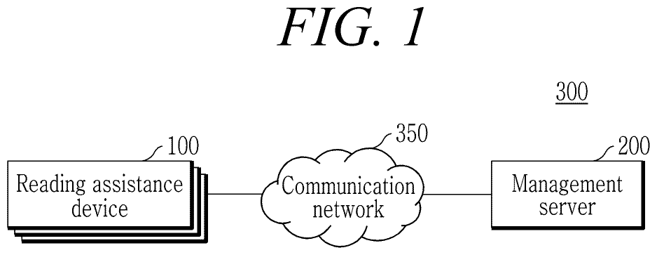

FIG. 1 is a configuration diagram illustrating a reading assistance system according to an embodiment of the present disclosure.

Referring to FIG. 1, the reading assistance system 300 provides a user (e.g. a reader) with visual and auditory effects corresponding to events occurring during a reading process to help the user become interested in reading. The reading assistance system 300 includes at least one reading assistance device 100 and a management server 200.

The reading assistance device 100 is a device that assists reading so that a user may become interested in reading books (e.g., paper books, audio books, e-books, etc.). The reading assistance device 100 analyzes the status of a user and the content of a book, and uses analyzed results to determine whether a preset event has occurred. When an event occurred, the reading assistance device 100 outputs at least one of a visual effect and an auditory effect corresponding to the event that occurred. Preferably, the reading assistance device 100 may provide a visual effect and an auditory effect to a user through augmented reality (AR), the change of brightness, the change of color, the change of background, the emphasis of a word, background sounds, sound effects, etc. Here, when a plurality of users uses one device, the reading assistance device 100 may support each user so that the user first performs a login procedure by using an ID and password before performing reading activities.

The management server 200 performs communication with the at least one reading assistance device 100, and to this end, a program or algorithm that is interfaced with the reading assistance device 100 is stored in the management server 200. The management server 200 manages the reading habit of a user who uses the reading assistance device 100. Specifically, the management server 200 receives monitoring information on a reading process generated by comprehensively analyzing information, such as the reading amount of a user, information on books which are completely read (e.g., book titles, authors, book contents, book genres, publishers, etc.), reading time, reading pattern, and interest levels in reading, from the reading assistance device 100. In addition, the management server 200 receives user information including the identification information, interest, age, gender, etc. of a user. The management server 200 may manage reading habit for each user by using the user information and the monitoring information which are received, and may support each user in checking the managed results. That is, when a plurality of users share the one reading assistance device 100, the management server 200 may distinguish each user through identification information and then may manage the reading habit of the corresponding user. The management server 200 may provide feedback to a user regarding his or her reading habits, when the user among the plurality of users is detected to have a relatively poorer reading habit than other users. For example, when the management server 200 detects a user who reads relatively less than other users, the management server 200 may suggest an additional reading amount and schedule information for reading time to that user. In addition, the management server 200 may process the monitoring information to generate new content. Here, the new content, which is image-based, user-tailored digital content consisting of new stories in which a user is included as a character, may be generated by using information from books that the user has read so far. The management server 200 transmits the generated content to the reading assistance device 100 so that the corresponding user may view the user-tailored content.

Meanwhile, the reading assistance system 300 establishes a communication network 350 between the reading assistance device 100 and the management server 200 to support communication therebetween. The communication network 350 may be composed of a backbone network and a subscriber network. The backbone network may be composed of one or more integrated networks of an X.25 network, a frame relay network, an ATM network, a multi-protocol label switching (MPLS) network, and a generalized multi-protocol label switching (GMPLS) network. The subscriber network may be fiber to the home (FTTH), asymmetric digital subscriber line (ADSL), cable network, zigbee, bluetooth, wireless LAN (IEEE 802.11b, IEEE 802.11a, IEEE 802.11g, IEEE 802.11n), wireless Hart (ISO/IEC62591-1), ISA 100.11a (ISO/IEC 62734), constrained application protocol (CoAP), message queuing telemetry transport (MQTT), wireless broadband (WIBro), Wimax, 3G, high speed downlink packet access (HSDPA), 4G, 5G, and 6G. In some embodiments, the communication network 350 may be an Internet network or a mobile communication network. In addition, the communication network 350 may include any other wireless or wired communication method which has been widely known or will be developed in the future.

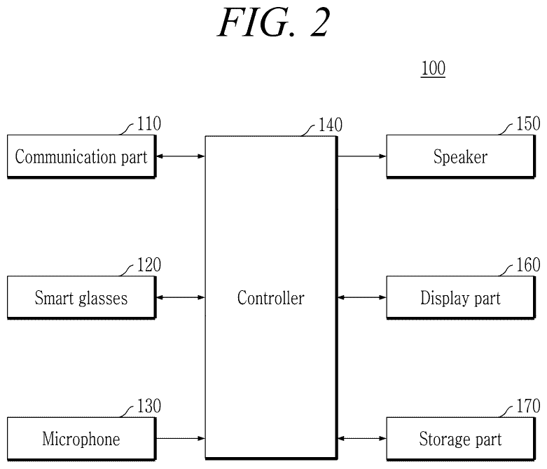

FIG. 2 is a block diagram illustrating the reading assistance device according to an embodiment of the present disclosure.

Referring to FIGS. 1 and 2, the reading assistance device 100 includes smart glasses 120 and a controller 140, and may include a communication part 110, a microphone 130, a speaker 150, a display part 160, and a storage part 170.

The communication part 110 performs communication with the management server 200. The communication part 110 transmits the user information and reading-related information to the management server 200. The communication part 110 receives content based on reading-related feedback information and the monitoring information from the management server 200.

The smart glasses 120, which are a pair of glasses worn on the face of a user, collect various pieces of information and provide augmented reality content. Specifically, when worn by a user, the smart glasses 120 capture an image of the front view of the user and simultaneously capture an image of the pupils of the user to generate corresponding image information. In other words, the smart glasses 120 may capture an image of a book that a user is reading while capturing an image of the pupils of the user looking at the book. For this purpose, the smart glasses 120 may include at least two cameras. In addition, the smart glasses 120 output augmented reality content provided when an event occurs. That is, the smart glasses 120 have a structure in which images (e.g., letters, shapes, numbers, etc.) may be output inside at least one of the transparent binocular lenses, thereby providing a three-dimensional visual effect to a user.

The microphone 130 receives the voice of a user. When the user reads a book by producing sound, the microphone 130 receives the corresponding voice. In addition, the microphone 130 receives the corresponding voice when the user vocalizes for a user input so as to use reading assistance service.

The controller 140 performs overall control of the reading assistance device 100. When a user reads a paper book, the controller 140 helps the user read parts of the paper book, which the user finds difficult, more easily while maximizing the tactile sensation of holding the paper book. In addition, the controller 140 serves to maximize the digital effect of an e-book while simultaneously outputting the e-book when a user reads the e-book, thereby amplifying the interest of the user in reading.

The controller 140 may perform the login procedure of a user for performing the reading assistance service by using a user input received from at least one of the smart glasses 120, the microphone 130, and the display part 150. For example, the controller 140 may verify biometric information (e.g., including voice tone, heart rate, fingerprint, iris, etc.) included in the user input and may support the login procedure on the basis of the verified biometric information. In addition, the controller 140 determines that a preset event has occurred when a preset word is included in book-related information (e.g., a paper book, an e-book, a book title, an author, a publisher, etc.) and the content of the part of a book currently being read by a user on the basis of the image information generated from the smart glasses 120. When the controller 140 determines that the event has occurred, the controller 140 controls the output of at least one of a visual effect and an auditory effect corresponding to the event.

Specifically, the controller 140 may collect book-related information by using at least one of a book cover, table of contents, author's note, and the part of a book currently being read by a user, included in the image information captured from the book. The controller 140 uses the collected book-related information to detect the content of the part of the book that the user is currently reading, and determines that a preset event has occurred when a specific word is included in the detected content. The specific word may be a word representing a specific name, a specific region, a specific action, etc., and the preset event may be an action that provides augmented reality content related to the specific word, provides a sound effect or background sound related to the specific word, provides an image related to the specific word, or adjusts the volume or speed of an output sound (e.g. music, voice, etc.).

In addition, the controller 140 determines that a reading completion event has occurred when the detected part of the book is the last page of the book, and controls a congratulatory message and information about the process of the reading to be output. In this case, the controller 140 may control the reading completion event to occur only when a record of a user reading the book sequentially from the first page of the book to the last page thereof is saved. Here, the controller 140 may generate the monitoring information on the reading process by comprehensively analyzing information such as information on the book that has been completely read (e.g., a book title, an author, a book content, a book genre, a publisher, etc.), reading time, a reading pattern, frequency of reading assistance, and reading interest. In addition, the controller 140 may estimate the area of interest by determining the likes and dislikes of words or sentences in a book on the basis of time for which the gaze of the user remains thereon, and may generate the monitoring information by further using the estimated area of interest. Preferably, the controller 140 may use the monitoring information to recommend reading habit that the user should supplement in the next reading and suggest actions to perform the recommended reading habit. For example, the controller 140 may suggest moving the reading time of a user to time before dinner when the user reads during an after-dinner time period and the concentration level of the user on reading drops sharply after 30 minutes of reading. Alternatively, the controller 140 may suggest reading recommendations for science books related to humanities when a user reads only humanities books.

The controller 140 detects status information about the gaze and focus of a user on the basis of the image information of the pupils of the user whose image is captured. The controller 140 may detect the status information on the basis of fluctuations of gaze and focus of the user. Here, the controller 140 may estimate the number of letters read per minute through the gaze of the user. The controller 140 uses the detected status information to calculate a reading-related concentration level. Here, the concentration level is a numerical expression of ability to concentrate while reading. When the concentration level is low, it may be determined that interest in reading is low, and when the concentration level is high, it may be determined that interest in reading is high. Accordingly, when the calculated concentration level is lower than a preset reference, the controller 140 may determine that an event has occurred and may control at least one of a visual effect and an auditory effect to be output. For example, when a user is drowsy and has a concentration level lower than the preset reference, the controller 140 may output a sound effect or turn on a light to improve the concentration level of the user. The controller 140 may use the status information to further detect the reading pattern of the user and whether the user has dyslexia. That is, the controller 140 may detect a reading pattern such as sequential reading or skimming, and detect whether a user has dyslexia through the pattern of re-reading words or sentences that have been read. The controller 140 supports chatbot conversations in conjunction with the smart glasses 120 on the basis of the concentration level, reading pattern, and the presence/absence of dyslexia which have been detected. The controller 140 supports so that the chatbot conversations may be conducted through written or spoken language and user-customized conversations may be conducted through self-learning capabilities during conversation.

When a user reads a book by producing sound, the controller 140 detects information related to at least one of pronunciation, a breathing position, a misread portion, and reading speed included in voice information about the sound. The controller 140 may collect the detected information and build a database for each user. In addition, the controller 140 provides feedback information on a part requiring correction on the basis of the detected information. After a user has completely read a paragraph, the controller 140 may provide feedback on the corresponding paragraph. When the pronunciation, breathing position, and reading in the corresponding paragraph are incorrect, the controller 140 may control a correct reading voice for the corresponding portion to be output. In addition, when the reading speed increases or decreases during the reading of a book, the controller 140 may control a sound effect or a notification message, which informs a user that the reading speed needs to be adjusted, to be output.

When reading an e-book that provides the contents of the book in an image form, the controller 140 replaces the name of at least one character of characters included in the contents of the book with a random name and controls the contents of the e-book to be output with the replaced name. In this case, the random name may be set by user input. That is, a user may set his or her name instead of the name of the character, giving the user the feeling of being the character in the book. In this case, the controller 140 may control an event to occur whenever a preset random name is output in the contents of an e-book, and at least one of a visual effect and an auditory effect related to the corresponding event to be output. For example, when the content of “Hong Gil-dong jumps toward the sky” is output while the name “Hong Gil-dong” is set, the controller 140 may control the image of Hong Gil-dong jumping toward the sky to be expressed in augmented reality while the letter color of Hong Gil-dong is emphasized in the image. In addition, the controller 140 performs the analysis of the contents of the book and uses the result of the analysis to estimate the personality of each of the characters. In this case, when a user input is input from a user and one of the characters is selected, the controller 140 may control the personality of the corresponding character to be reflected in a chatbot conversation pattern. For example, the controller 140 supports chatbot conversations to proceed with a conversation pattern that includes the speech patterns, dialects, etc. of the selected character. Through this, the controller 140 may create an immersive experience that allows a user to be more immersed in the corresponding book. When voice information is input from a user and a specific word is included in the voice information, the controller 140 controls the e-book to return to a preset page. The controller 140 controls a paragraph or word related to the specific word in the returned page to be highlighted, thereby helping the user to intuitively check information about the specific word even on the returned page. In addition, when touch input information is input at the location of an illustration or character, the controller 140 outputs an effect, such as a sound effect, a background sound, a voice, and screen transition, corresponding to the illustration or character located at the corresponding touch input information.

Meanwhile, when at least one of the reading-related feedback information and content is received from the management server 200, the controller 140 controls the corresponding feedback information and the content so that a user may check the corresponding feedback information and the content. That is, the controller 140 may inform a user of improvements to his/her reading habit through the feedback information and may stimulate the interest of the user in reading through the content.

The speaker 150 outputs sounds performed during the reading assistance process. The speaker 150 outputs a background sound, a sound effect, etc. that provide an auditory effect. In addition, the speaker 150 outputs the contents of an e-book when the user reads the e-book.

The display part 160 outputs a screen that is performed during the reading assistance process. The display part 160 displays a book cover, illustrations, book contents, etc. when a user reads an e-book. In addition, the display part 160 outputs visual effects such as a brightness change, a color change, and a background change of e-books. The display part 160 may include a display including a touch screen function, and the display may collect the touch input information of a user through the touch screen function. Preferably, the display may include at least one of a liquid crystal display (LCD), a thin film transistor-liquid crystal display (TFT LCD), an organic light-emitting diode (OLED), a flexible display, and a 3D display.

The storage part 170 stores a program or algorithm for operating the reading assistance device 100. The storage part 170 stores information about e-books that a user has read previously, is currently reading, or will read in the future. The storage part 170 stores information about each event and information about visual and auditory effects output for each event. The storage part 170 stores the feedback information and content received from the management server 200. The storage part 170 may include at least one storage medium among a flash memory type, a hard disk type, a multimedia card micro type, a card type memory (for example, an SD or XD memory, etc.), a random access memory (RAM), a static random access memory (SRAM), a read-only memory (ROM), an electrically erasable programmable read-only memory (EEPROM), a programmable read-only memory (PROM), a magnetic memory, a magnetic disk, and an optical disk.

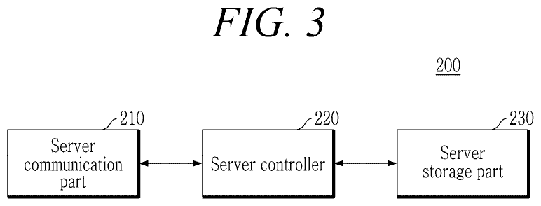

FIG. 3 is a block diagram illustrating the management server according to an embodiment of the present disclosure.

Referring to FIGS. 1 and 3, the management server 200 may include a server communication part 210, a server controller 220, and a server storage part 230.

The server communication unit 210 performs communication with the at least one reading assistance device 100. The server communication part 210 receives the user information and the reading-related information from the reading assistance device 100. The server communication part 210 transmits content based on the reading-related feedback information and the monitoring information to the reading assistance device 100.

The server controller 220 performs overall control of the management server 200. The server controller 220 manages the reading habit of each user on the basis of the user information and the monitoring information of the user. That is, the server controller 220 may manage reading habits for a plurality of users. To this end, the server controller 220 detects the reading habit of each user and uses the detected reading habit to diagnose deficiencies by comparing the reading habit with those of other users. The server controller 220 generates feedback information by deriving alternatives for the diagnosed deficiencies. The server controller 220 helps the corresponding user check the generated feedback information to improve his/her reading habit.

The server controller 220 processes the monitoring information on the basis of the user information and generates a new story in which a user included in the user information is included as a character. Preferably, the server controller 220 may generate a new story by using artificial intelligence technology, but is not limited thereto. The server controller 220 generates image-based content by using the generated story. Here, the content may be content made up of connected images, such as illustrations for a story, or content made up of images connected in sequence, such as a video. The server controller 220 transmits the content to the reading assistance device that transmitted the user information and supports the content to be output from the reading assistance device.

The server storage part 230 stores a program or algorithm for operating the management server 200. The server storage part 230 stores information about each reading assistance device, user information about each user, and monitoring information. In addition, the server storage part 230 stores content based on the monitoring information. The server storage part 230 may include at least one storage medium among a flash memory type, a hard disk type, a multimedia card micro type, a card type memory (e.g., an SD or XD memory, etc.), RAM, SRAM, ROM, EEPROM, PROM, a magnetic memory, a magnetic disk, and an optical disk.

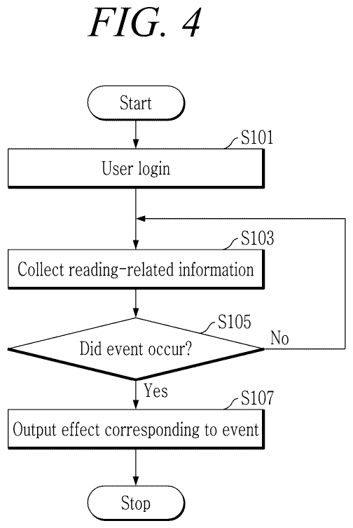

FIG. 4 is a flowchart illustrating a reading assistance method according to an embodiment of the present disclosure.

Referring to FIGS. 1 and 4, in the reading assistance method, image information obtained by capturing the reading process of a user is used to determine an event occurring during the reading process, and when the event occurs, at least one of a visual effect and an auditory effect corresponding to the event is output, thereby stimulating the interest of the user in reading. Through this, the reading assistance method may improve the literacy, thinking skill, and creativity of a user by encouraging continuous reading activity.

In S101, the reading assistance device 100 performs a login procedure for a user who is reading. The reading assistance device 100 may perform the login procedure by using biometric information, ID/password information, etc. Through this, the reading assistance device 100 supports a plurality of users to receive reading assistance service with one device.

In S103, the reading assistance device 100 collects reading-related information. The reading assistance device 100 collects book-related information by using at least one of a book cover, a table of contents, an author's note, and a portion of a book currently being read by a user, which are included in the image information of a book captured. In addition, the reading assistance device 100 collects the book-related information by using voice information spoken by a user and touch input information input by the touch of the user. Through this, the reading assistance device 100 may collect information about an image that a user views, a voice with which the user speaks, and an action that the user performs.

In S105, the reading assistance device 100 determines whether an event has occurred. The reading assistance device 100 determines whether the contents of the portion of the book include a preset word on the basis of the book-related information and the portion of the book that a user is currently reading. When the preset word is included, the reading assistance device 100 determines that an event has occurred and performs S107. When the preset word is not included, the reading assistance device 100 determines that an event has not occurred and re-performs S103.

In S107, the reading assistance device 100 outputs an effect corresponding to the event. The reading assistance device 100 outputs at least one of a visual effect and an auditory effect corresponding to the occurred event. In this case, the visual effects may include augmented reality, a brightness change, a color change, a background change, word emphasis, etc., and the auditory effect may include a background sound, a sound effect, etc.

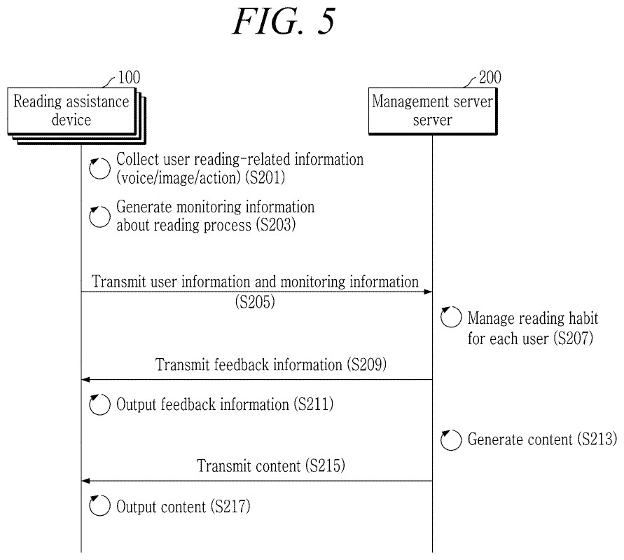

FIG. 5 is a flowchart illustrating a reading assistance method according to another embodiment of the present disclosure.

Referring to FIG. 5, the reading assistance method helps a user be interested in reading by generating new content on the basis of his or her reading habit and providing the generated content to the user.

In S201, the reading assistance device 100 collects reading-related information of the user. The reading assistance device 100 collects the book-related information by using at least one of a book cover, a table of contents, an author's note, and the portion of the book currently being read by a user, which are included in the image information of the book captured. In addition, the reading assistance device 100 collects the book-related information by using voice information spoken by a user and touch input information input by the touch of the user. Through this, the reading assistance device 100 may collect information about an image that a user views, a voice with which the user speaks, and an action that the user performs.

In S203, the reading assistance device 100 generates monitoring information about the reading process. The reading assistance device 100 generates the monitoring information by using information generated during the reading process of a user. The reading assistance device 100 may generate the monitoring information about the reading process by comprehensively analyzing information such as information about a book that has been completely read (e.g., a book title, an author, a book content, a book genre, a publisher, etc.), reading time, a reading pattern, frequency of reading assistance, and reading interest.

In S205, the reading assistance device 100 transmits the user information and the monitoring information to the management server 200. Here, the user information may be information about a user who is currently performing reading activity through the reading assistance device 100.

In S207, the management server 200 manages reading habit for each user. The management server 200 may classify the monitoring information of each user on the basis of the user information and manage the reading habit of the corresponding user by using the classified monitoring information. When a user among a plurality of users has reading habit that is relatively unbalanced compared to other users, the management server 200 detects the deficiency of the user on the basis of the unbalanced reading habit. The management server 200 may generate feedback information about the detected deficiency and provide the generated feedback information to the corresponding user.

In S209, the management server 200 transmits the feedback information to the reading assistance device 100 used by the corresponding user.

In S211, the reading assistance device 100 outputs the feedback information. The reading assistance device 100 provides the feedback information to the user to help the user improve his or her reading habit.

In S213, the management server 200 processes the monitoring information to generate new content. The management server 200 processes the monitoring information on the basis of the user information and generates a new story in which the user is included as a character. The management server 200 generates image-based content by using the generated story.

In S215, the management server 200 transmits the content to the reading assistance device 100 used by the corresponding user.

In S217, the reading assistance device 100 outputs the content. The reading assistance device 100 provides the content to the user to help the user be interested in reading.

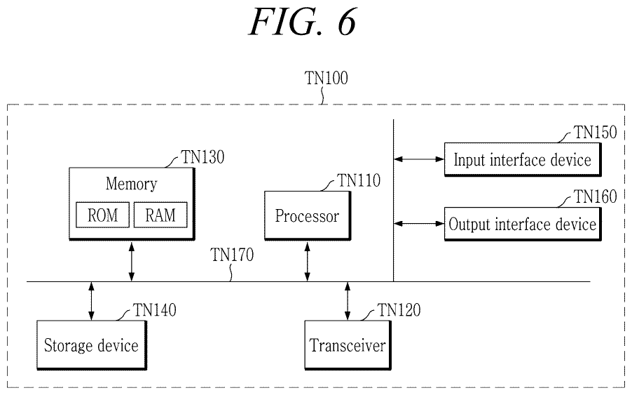

FIG. 6 is a block diagram illustrating a computing device according to an embodiment of the present disclosure.

Referring to FIG. 6, the computing device TN100 may be a device described in this specification (e.g., the reading assistance device, the management server, etc.).

The computing device TN100 may include at least one processor TN110, a transceiver TN120, and a memory TN130. In addition, the computing device TN100 may further include a storage device TN140, an input interface device TN150, and an output interface device TN160. Components included in the computing device TN100 may be connected to each other by a bus TN170 and may communicate with each other.

The processor TN110 may execute a program command stored in at least one of the memory TN130 and the storage device TN140. The processor TN110 may mean a central processing unit (CPU), a graphics processing unit (GPU), or a dedicated processor in which methods according to embodiments of the present disclosure are performed. The processor TN110 may be configured to implement procedures, functions, and methods described in connection with embodiments of the present disclosure. The processor TN110 may control each component of the computing device TN100.

Each of the memory TN130 and the storage device TN140 may store various information related to the operation of the processor TN110. Each of the memory TN130 and the storage device TN140 may be configured as at least one of a volatile storage medium and a non-volatile storage medium. For example, the memory TN130 may be configured as at least one of read-only memory (ROM) and random access memory (RAM).

The transceiver TN120 may transmit or receive wired or wireless signals. The transceiver TN120 may be connected to a network and perform communications.

Meanwhile, the embodiments of the present disclosure are not implemented only through the devices and/or method described so far, and may be implemented through a program that realizes a function corresponding to the configuration of the embodiments of the present disclosure or a recording medium on which the program is recorded, and such implementation may be easily implemented by those skilled in the art in the technical field to which the present disclosure belongs from the description of the embodiments described above.

Although the embodiments of the present disclosure have been described in detail above, the scope of the rights of the present disclosure is not limited thereto, and various modifications and improvements made by those skilled in the art using the basic concept of the present disclosure defined in the following claims also fall within the scope of the rights of the present disclosure.

What is claimed is:

1. A reading assistance device comprising:
smart glasses that are worn by a user and capture images of a book which the user is reading and the pupils of the user who is reading to generate corresponding image information; and
a controller that detects book-related information and a portion of the book that the user is currently reading on the basis of the generated image information, determines that a preset event has occurred when contents of the detected portion of the book include a preset word, and controls at least one of a visual effect and an auditory effect corresponding to the event to be output.

2. The reading assistance device of claim 1, wherein the controller detects status information about a gaze and a focus of the user on the basis of the image information of the pupils of the user whose image is captured, calculates a reading-related concentration level by using the detected status information, and determines that the event has occurred when the calculated concentration level is lower than a preset reference.

3. The reading assistance device of claim 2, wherein the controller further detects a reading pattern of the user and whether the user has dyslexia by using the status information, and supports chatbot conversation in conjunction with the smart glasses on the basis of the detected concentration level, the reading pattern, and whether the user has dyslexia.

4. The reading assistance device of claim 1, wherein when the user reads the book by producing sound, the controller detects information related to at least one of pronunciation, a breathing position, reading speed, and a misread portion included in voice information about the sound, and provides feedback information on a part requiring correction on the basis of the detected information.

5. The reading assistance device of claim 1, wherein when reading an e-book that provides contents of the book in an image form, the controller replaces a name of at least one character of characters included in the contents of the book with a random name and controls the contents of the e-book to be output with the replaced name.

6. The reading assistance device of claim 5, wherein whenever the random name is output in the contents of the e-book, the controller determines that the event has occurred.

7. The reading assistance device of claim 5, wherein the controller performs analysis of the contents of the book, uses a result of the analysis to estimate a personality of each of the characters, and, when one of the characters is selected, controls a personality of the corresponding character to be reflected in a chatbot conversation pattern.

8. The reading assistance device of claim 5, wherein when a user input is input from the user and the user input includes a specific word, the controller allows the e-book to return to a preset page thereof.

9. The reading assistance device of claim 1, wherein the controller determines that a reading completion event has occurred when the detected portion of the book is a last page of the book, and controls a congratulatory message and information about a process of the reading to be output.

10. The reading assistance device of claim 9, wherein the controller controls the reading completion event to occur only when a record of the user reading the book sequentially from a first page of the book to the last page thereof is saved.

11. A reading assistance method performed by a reading assistance device, the reading assistance method comprising:
generating corresponding image information by capturing a book being read by a user and the pupils of the user reading the book;
detecting book-related information and a portion of the book that the user is currently reading on the basis of the generated image information; and
outputting at least one of a visual effect and an auditory effect corresponding to a preset event by determining that the event has occurred when contents of the detected portion of the book include a preset word.

12. A reading assistance system comprising:
at least one reading assistance device that assists a reading process so that a user is interested in reading; and
a management server that receives user information of the user and monitoring information about the reading process the user from the reading assistance device and manages reading habit of the user by using the received monitoring information,
wherein the reading assistance device comprises:
smart glasses that are worn by the user and capture images of a book which the user is reading and the pupils of the user who is reading to generate corresponding image information; and
a controller that detects book-related information and a portion of the book that the user is currently reading on the basis of the generated image information, determines that a preset event has occurred when contents of the detected portion of the book include a preset word, and controls at least one of a visual effect and an auditory effect corresponding to the event to be output.

13. The reading assistance system of claim 12, wherein the management server processes the monitoring information on the basis of the user information to generate a new story in which the user is included as a character, and generates image-based content by using the generated story.

14. The reading assistance system of claim 12, wherein when the user reads the book by producing sound, the controller detects information related to at least one of pronunciation, a breathing position, reading speed, and a misread portion included in voice information about the sound, and provides feedback information on a part requiring correction on the basis of the detected information.

15. The reading assistance system of claim 12, wherein when reading an e-book that provides contents of the book in an image form, the controller replaces a name of at least one character of characters included in the contents of the book with a random name and controls the contents of the e-book to be output with the replaced name.
